(19) United States
(12) Patent Application Publication
(10) Pub. No.: US 2010/0203608 A1
(43) Pub. Date: Aug. 12, 2010

(54) METHOD FOR THE CONVERSION OF BIOMASS FROM RENEWABLE RAW MATERIALS IN ANAEROBIC FERMENTERS

(75) Inventors: Bjoern Schwarz, Dresden (DE); Burghard Fassauer, Dresden (DE); Eberhard Friedrich, Radebeul (DE); Hannelore Friedrich, Radebeul (DE); Alexander Michaelis, Dresden (DE)

Correspondence Address: GREENBLUM & BERNSTEIN, P.L.C., 1950 ROLAND CLARKE PLACE, RESTON, VA, 20191, US

(73) Assignee: Fraunhofer-Gesellschaft Zur Foerderung Der Angewandten Forschung E.V., Muenchen (DE)

(21) Appl. No.: 12/671,376

(22) PCT Filed: Jul. 23, 2008
(86) PCT No.: PCT/EP08/59677

§ 371 Date: Apr. 16, 2010

(30) Foreign Application Priority Data
Jul. 30, 2007 (DE) 10 2007 037 202.9

(51) Int. Cl. C12P 5/02

(52) U.S. Cl. 435/167

(57) ABSTRACT

The invention relates to the field of biochemistry and relates to a method that is used in a biogas production plant. The object of the present invention is a method which realizes a shorter total retention time of the raw materials in the biogas plant and/or a higher quantity and/or quality of the biogas. The object is attained through a method in which renewable raw materials are placed in an at least first anaerobic fermenter/reactor together with liquid and further starting materials necessary for methanogenesis, and there subjected to a fermentation process. Subsequently the fermentation residue is subjected to a solid/liquid phase separation and the separated solid phase is subjected to thermopressure hydrolysis at temperatures of at least 170° C. and pressures of at least 1.0 MPa. The solid phase thus treated may either be returned to the first anaerobic fermenter/reactor or fed to a second anaerobic fermenter/reactor and subjected to a further fermentation process.

### BACKGROUND

The invention relates to the fields of biochemistry and energy production and relates to a method for converting biomass from renewable raw materials to biogas in anaerobic fermenters, which is used in a biogas production plant, and the use of which is possible in the monofermentation of renewable raw materials as well as in co-fermentation with commercial fertilizers (e.g., liquid manure) in agricultural biogas plants or in co-fermentation with sewage in municipal sewage treatment plants.

The conversion of biomass into biogas to be energetically recovered, while utilizing the biochemical capacity of an anaerobic mixed population of microorganisms, is practiced on an industrial scale in agricultural biogas plants as well as in the digestion towers of municipal sewage treatment plants. The process engineering used thereby covers a very broad spectrum of combinations of the number and switching of fermenters, process temperature (mesophilic, thermophilic), substrate treatment, charging regime, intermixing, retention time and loading rate.

In the utilization of renewable raw materials as the main substrate or co-substrate for biogas production, the chemical structure thereof prevents a complete conversion into biogas. Large proportions of the plant material are composed of cellulose, hemicellulose and lignin hardly accessible or not accessible at all for microorganisms. This leads to unsatisfactory and in part uneconomical degradation ratios with the application of conventional fermentation technologies, which apart from a mechanical coarse grinding of the input material, do not carry out any further substrate treatment. At 50 to 150 days, the retention times of the substrates in anaerobic fermenters are very long and the energy yield in the form of biogas is nevertheless unsatisfactory.

Furthermore, with reactors switched in series (cascades) only the first reactor is utilized to full capacity, since the largest proportion of the microbiologically available organic substances are already converted in the first 20 to 30 days. All of the downstream reactors are very limited in their degradation activity and speed. The reason for this is the very slow hydrolysis of the remaining organic fractions. This degradation step limiting speed leads to an under-utilization of the methanogenesis, which still has marked reserves.

For better utilization of the energy contained primarily in renewable raw materials in fermentation, the hydrolysis, that is, the breakdown of complex macromolecules into their basic components, must be accelerated externally. A number of methods exist for this according to the prior art and primarily in the field of raw materials recovery from renewable raw materials (basic chemistry). Various combinations of mechanical pre-treatment (milling, grinding), physical (vapor explosion method, hot water treatment, thermo-pressure hydrolysis), chemical (acids, lyes, wet oxidation) and enzymatic methods are used, e.g., for a lysis of cellulose and hemicellulose. However, the cited methods cannot be used directly in the field of application of the biogas generator, since, for example, the need for energy and chemicals for the volume flows to be treated is uneconomically high. In the fermentation or co-fermentation of renewable raw materials, according to the prior art the following methods are used for an intensification of the degradation:
Mechanical pre-degradation (e.g., by means of an extruder);
   High solids content of the treated substrate necessary→use before the first fermenter for solid substrates (silages)
   Low acceleration of the degradation in the first fermenter, which has a good degradation capacity even without substrate preparation
Enzyme dosage;
   Specialized for a few substrate groups (e.g., cellulose)
   Slight increase in the degree of degradation
   Relatively high cost of enzymes
Separate hydrolysis/methanation, solid/liquid separation and pH control through the recirculation of the liquid phase (EP 0 566 056 A1);
   Uncoupling of solid substances that are difficult to hydrolyze from the main flow
   Biological hydrolysis of the separated solids in extra fermenter→hydrolysis too slow and limited by restricted lysis capacity of the microorganisms
Thermal disintegration between first and second fermenters connected in series (DE 198 58 187 C2, DE 103 45 600 A1);
   10 to 120 min at 60 to 90° C.→temperatures too high to maintain methanogenic bacteria and too low for the lysis of the fractions that are difficult to degrade
Thermal disintegration as intermediate step (FR 2 711 980 C2);
   Full stream treatment at 80 to 175° C.→very high energy consumption and destruction of the methanogenic bacteria, and
Dewatering and heat treatment (EP 0 737 651 A1)
   At 60° C. or higher→temperatures too low for effective lysis.
      The disadvantages of the solutions from the prior art lie above all in the inadequate gas yield compared to the total retention time of the renewable raw materials in the biogas plant.

### SUMMARY OF THE INVENTION

The object of the present invention lies in disclosing a method for converting biomass from renewable raw materials to biogas in anaerobic fermenters, which realizes a shorter total retention time of the renewable raw materials in the biogas plant and/or a higher quantity and/or quality of the biogas.

The object is attained with the invention disclosed in the claims. Advantageous embodiments are the subject matter of the subordinate claims.

### BRIEF DESCRIPTION OF THE DRAWINGS

FIG. 1 shows the process description with two fermenters.

FIG. 2 shows the process description with one fermenter.

### DETAILED DESCRIPTION OF THE INVENTION

With the method according to the invention for converting biomass from renewable raw materials to biogas in anaerobic fermenters, renewable raw materials are placed in an at least first anaerobic fermenter/reactor together with liquid and further starting materials necessary for methanogenesis, and there subjected to a fermentation process. Subsequently the fermentation residue is subjected to a solid/liquid phase separation and the separated solid phase is subjected to thermopressure hydrolysis at temperatures of at least 170° C. and pressures of at least 1.0 MPa. The solid phase thus treated may either be returned to the first anaerobic fermenter/reactor or fed to a second anaerobic fermenter/reactor and subjected to a further fermentation process.

Advantageously, liquid manure and/or sewage sludges and/or process waters are added as further starting materials necessary for the methanogenesis.

Likewise advantageously, the solid/liquid phase separation is carried out by pressing, centrifuging or screening.

Furthermore advantageously, the thermopressure hydrolysis is supplemented by mechanical processing of the solid phase before or during the thermopressure hydrolysis.

It is also advantageous if the thermopressure hydrolysis is supplemented by chemical and/or enzymatic processing of the solid phase before or during the thermopressure hydrolysis.

It is also advantageous if the thermopressure hydrolysis is carried out at temperatures in the range of 170 to 250° C.

It is likewise advantageous if the thermopressure hydrolysis is carried out at pressures of 1 to 4 MPa.

It is furthermore advantageous if the solid phase is fed to a second anaerobic fermenter/reactor after the thermopressure hydrolysis, to which at least portions of the liquid phase are fed after the solid/liquid phase separation.

It is also advantageous if the thermopressure hydrolysis is carried out by utilizing the exhaust heat of other processes.

With the method according to the invention it is possible for the first time to disclose a biogas production method which produces gas in a shorter time and/or with a higher yield. The total retention time of the biomass can be reduced thereby from previously 50 to 150 days to less than 50 days. At the same time, the quantity as well as the quality of the biogas produced can be improved by a degradation degree of the biomass used of over 80%.

With the method according to the invention, renewable raw materials are processed to form biogas. Within the scope of the present invention, renewable raw materials means all raw materials that are cultivated for the purpose of energy production. Furthermore, this term also covers residues from agricultural production.

Sewage sludges are not a renewable raw material as defined by the present invention.

The difference between the renewable raw materials and sewage sludges for the method according to the invention is also to be seen in particular in that the renewable raw materials in each process step have a much higher proportion of organic dry residue, cellulose and lignin as well as a much larger particle size.

With the method according to the invention a virtually complete energetic utilization of the biomass used and the conversion thereof into biogas can be realized. Virtually the entire primary energy contained in the substrate can be converted into biogas, which is subsequently converted into electricity.

The existing fermenter volumes can also be used much more effectively and plants at the planning stage can be designed smaller due to the clearly increased degradation speed.

Virtually a complete utilization of the methanation capacity of the anaerobic fermenters is achieved with the method according to the invention. That means that all of the fermenters/reactors of a biogas plant for the fermentation or co-fermentation of renewable raw materials can always be charged with such a large quantity of easily converted substrates that a marked speed limitation no longer occurs through the hydrolysis. The substances supplied are converted so quickly that sufficient acetic acid is always available for the methane stage.

With an average retention time of 25 days in the first anaerobic fermenter/reactor, as is known, about 40% of the organic starting materials are degraded and converted into biogas. This first anaerobic fermenter/reactor thereby operates at full methanation capacity, which has been proven by a higher content of organic acids determined in practice as an intermediate product of the anaerobic degradation chain. In particular a higher content of acetic acid indicates either an inhibition of the methanogens utilizing the acetic acid or a high utilization of this group of bacteria.

In order to provide starting materials with a similarly high proportion of easily available substrates for a fermentation process in the downstream second anaerobic fermenter/reactor, according to the invention two intermediate steps are realized. Firstly, the fermentation residue of the first anaerobic fermenter/reactor is subjected to a solid/liquid phase separation. The fermentation residue is composed essentially of non-degraded renewable raw materials, microorganisms and water. Through a simple centrifugal force dewatering or through pressing, the water and the microorganisms are jointly separated and can be fed as a liquid phase to the second anaerobic fermenter/reactor or treated as a degradation product.

The likewise separated solid phase, which represents approx. 20 to 30% of the total volume of the fermentation residue of the first anaerobic fermenter/reactor, contains essentially the gas-forming potential still available, which can be released through a virtually complete splitting of the macromolecules that are very difficult to hydrolyze, in the thermopressure hydrolysis step in the second anaerobic fermenter/reactor.

The thermopressure hydrolysis process according to the invention is then carried out, for example, in an additional reactor or in the pipeline between a first and second anaerobic fermenter/reactor. Advantageously, the exhaust heat of a combined heat and power plant can be used for this purpose. The very large energy quantities are fed to the solid phase, for example, by means of an exhaust heat exchanger and the solid phase subjected in batch operation to the thermopressure hydrolysis at least 170° C. and 1.0 MPa for between 10 and 120 min. An additional support of the thermopressure hydrolysis can be achieved by mechanical movement of the solid phase. Through the thermopressure hydrolysis at relatively high temperatures, the celluloses essentially contained in the solid phase and lignins from the fermentation residue after the phase separation are broken down and degraded into their basic constituents (monomers) and thus made accessible for further conversion to biogas in a fermenter/reactor. This has not hitherto been possible with the solutions of the prior art.

The solid phase thus treated is fed in full to the second anaerobic fermenter/reactor where it is converted into biogas virtually completely and at a comparatively high speed.

In the event that only one anaerobic fermenter/reactor is present, the solid phase subjected to the thermopressure hydrolysis can also be returned to this reactor again. This means that essentially only a batchwise process can be carried out. However, one advantage of this solution is the reduced quantity of fresh biomass required as a starting material.

However, the method according to the invention can also be realized inside a plurality of fermenter/reactors, if they are already present, for example.

Furthermore, through the solid/liquid phase separation the advantage is achieved that the treatment volume is greatly reduced, which means a saving or more effective utilization of the thermal energy used. Furthermore, the methane bacteria and other microorganisms intact in the fermentation residue after the first anaerobic fermenter/reactor are not subjected to a heat treatment and can further be used in the first or second anaerobic fermenter/reactor.

Another advantage lies in the increase of the hydraulic retention time in the second reactor, since part of the liquid phase can be withdrawn from the total process.

A further advantage of the method according to the invention lies in its high flexibility, which results from the solid/liquid phase separation. The environment conditions of the second anaerobic fermenter/reactor can be influenced by changing the liquid phase addition, which has a positive effect on the operating stability and thus the biogas yield.

The invention is explained in more detail below based on an exemplary embodiment.

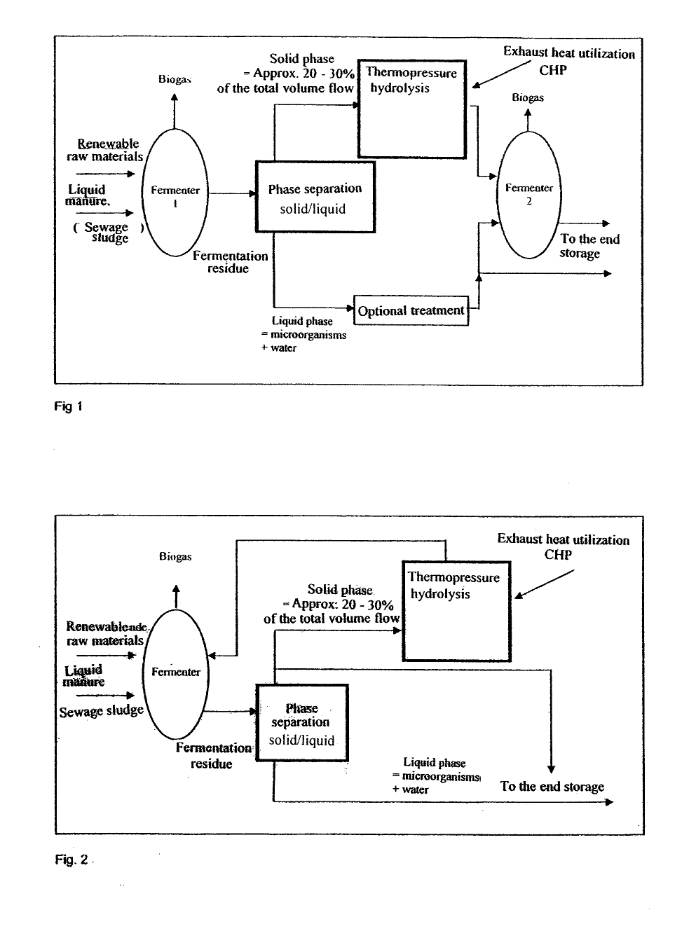

They show:
FIG. 1 The process description with two fermenters
and
FIG. 2 The process description with one fermenter.

50 t silage, composed of 60% corn and 40% rye, is fed daily together with 100 m³ liquid manure to a continuously operated anaerobic stirred-tank digester as a first fermenter/reactor. In this first fermentation step within 20 days hydraulic retention time approx. 40% of the organic substrates contained in the input is converted to biogas.

The fermentation residue of this first stage (approx. 136 t/d) is dewatered with the aid of a screw press. This produces approx. 107 t liquid phase with a solid content of 4% and approx. 31 t solids with a dry residue of 30%. Two thirds of the liquid phase are transferred to the following fermenter 2, the other third to the final deposition (material recycling). The separated solids are subjected to a 30-minute treatment at 220° C. and 2.5 MPa. This is carried out in batch operation utilizing the exhaust heat of a combined heat and power plant (CHP). Following this thermopressure hydrolysis, the partially liquefied solids likewise reach the second fermenter/reactor. The organic portions contained are converted into biogas almost completely in the second fermenter/reactor during the hydraulic retention time of 20 days. The total degree of degradation of the organic substance achieved in the two fermenters is 80% in all.

1. A method for converting biomass from renewable raw materials to biogas in an anaerobic fermenter/reactor, comprising:
placing renewable raw materials in an at least first anaerobic fermenter/reactor together with liquid and other starting materials necessary for methanogenesis;
subjecting the renewable raw materials, liquid, and other starting materials to a fermentation process, thereby yielding a fermentation residue;
subsequently subjecting the fermentation residue to a solid/liquid phase separation;
subjecting the solid phase to thermopressure hydrolysis at a temperature of at least 170° C. and a pressure of at least 1.0 MPa; and
either
guiding the solid phase thus treated back into the first anaerobic fermenter/reactor, or
feeding the solid phase thus treated to a second anaerobic fermenter/reactor and subjecting it to a further fermentation process.

2. The method according to claim 1, in which liquid manure and/or sewage sludges and/or process waters are added as further starting materials necessary for the methanogenesis.

3. The method according to claim 1, in which the solid/liquid phase separation is carried out by pressing, centrifuging or screening.

4. The method according to claim 1, in which the thermopressure hydrolysis is supplemented by mechanical processing of the solid phase before or during the thermopressure hydrolysis.

5. The method according to claim 1, in which the thermopressure hydrolysis is supplemented by chemical and/or enzymatic processing of the solid phase before the thermopressure hydrolysis.

6. The method according to claim 1, in which the thermopressure hydrolysis is carried out at a temperature in the range of 170 to 250° C.

7. The method according to claim 1, in which the thermopressure hydrolysis is carried out at a pressure of 1 to 4 MPa.

8. The method according to claim 1, in which the solid phase is fed to a second anaerobic fermenter/reactor after the thermopressure hydrolysis, to which at least portions of the liquid phase are fed after the solid/liquid phase separation.

9. The method according to claim 1, in which the thermopressure hydrolysis is carried out by utilizing the exhaust heat of other processes.
